(19) United States
(12) Patent Application Publication
(10) Pub. No.: US 2016/0296426 A1
(43) Pub. Date: Oct. 13, 2016

(54) SOAP ASSEMBLY

(71) Applicant: Priscila Yazbek, Austin, TX (US)

(72) Inventor: Priscila Yazbek, Austin, TX (US)

(21) Appl. No.: 14/685,207

(22) Filed: Apr. 13, 2015

(51) Int. Cl. A61K 8/02; A41G 1/00; A61Q 19/10

(52) U.S. Cl. CPC A61K 8/0216; A61Q 19/10; A41G 1/001

(57) ABSTRACT

Embodiments include an assembly of single use soap elements to form a decorative source for single use soaps.

### BACKGROUND

Maintaining a certain level of hygiene is important in preventing the spread of diseases. A group of organic compounds comprising salts of fatty acids, when dissolved in water, facilitates cleaning away microorganisms from a surface such as the human skin. Such a group of organic compounds are generally referred to as a “soap” or “cleansing agent”.

Traditional soap receptacles are generally simple constructions formed of ceramic, glass, or other water impervious material and are designed to receive a multi-use soap or bar. There are a myriad of designs of such soap receptacles incorporating different shapes and ornamental surface treatment. Soap receptacles share a common limitation in that their water impervious material tends to collect and retain moisture from soap that is returned to the receptacle wet after it has been used. The soap erodes as it rests in the associated water and it leaves an unsightly residue on the soap receptacle that tends to build up after repeated use and becomes an eyesore.

Thus, there is a need for additional devices or arrangement for presenting soap as a decorative item and reducing the residue and mess associated with multiple use soap articles.

### SUMMARY

Embodiments of the invention are directed to an assembly of single use soap elements. The single use soap elements can be assembled into decorative articles that can be placed near a sink, tub, or shower. In certain aspects a soap assembly can be provided. In further aspects include an assembly kit that includes a base having assembly grooves and a plurality of single use soap elements for assembly. In a further aspect the single use soap elements are configured to be assembled in the groves of the base to form a decorative item by being placed in the grooves formed in the base forming a soap assembly. In certain aspects the soap assembly forms a flower. In certain aspects the soap elements may be a scented soap elements.

In certain embodiments a soap assembly comprises a base and a plurality of single use soap elements, wherein the base forms grooves to align the single use soap elements into a decorative assembly. In certain aspects the single use soap elements are in the shape of flower petals. In a further aspect the decorative assembly is in the shape of a flower, e.g., a rose. In certain aspects the single use soap elements are approximately 1, 2, 3, 4 to 3, 4, 5, 6 cm in diameter, including all values and ranges there between. The single use soap elements can be about 0.5, 1, 2, 3, 4, 5, 6 to 5, 6, 7, 8, 9, 10 millimeters thick, including all values and ranges there between. In certain respects single use soap elements may vary in size within a single assembly and may be bent or the shape altered as needed to form the assembly.

Other embodiments are directed to methods for assembling a plurality of soap elements into a soap assembly. The method includes arranging the soap elements of a kit by positioning the soap elements in a groove in a base.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustrates a perspective view of one example of a soap assembly.

FIG. 2. Illustrates a top view of one example of a soap assembly.

FIG. 3. Illustrates a bottom view of one example of a soap assembly.

FIG. 4. Illustrates a side view of one example of a soap assembly.

FIG. 5. Illustrates a base used to assemble one example of a soap assembly.

FIG. 6. Illustrates one example of assembling the soap assembly, this example showing the positioning of a first outer layer.

FIG. 7. Illustrate a view of one example of a soap assembly partially constructed and having a first outer layer and the center assembled.

### DESCRIPTION

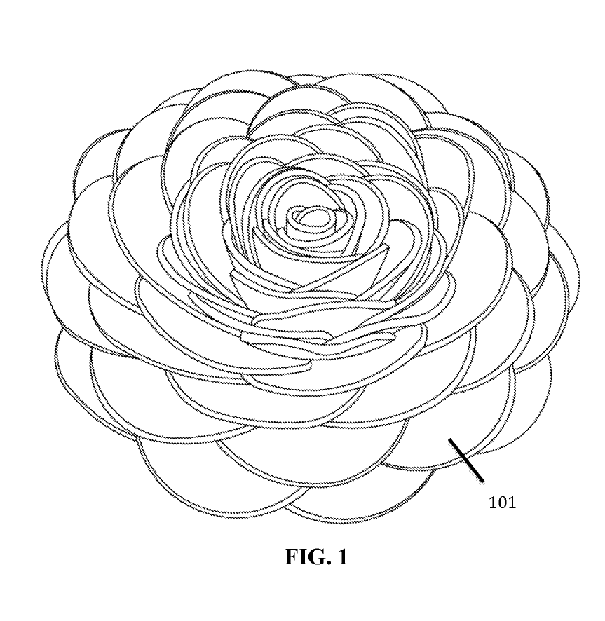

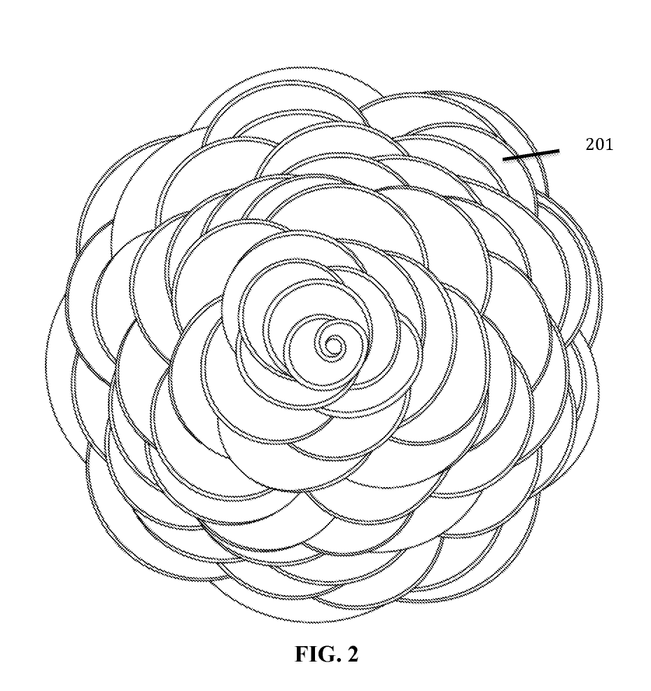

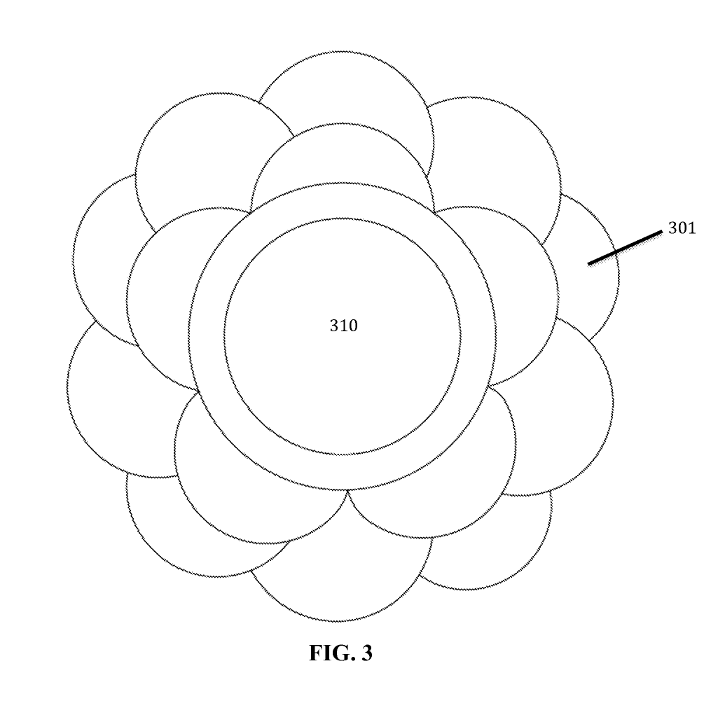

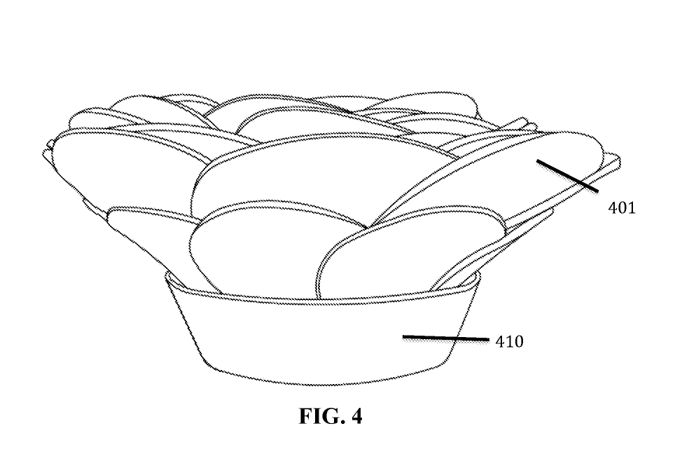

FIG. 1 illustrates a perspective view of an example of a soap assembly described herein. In this example single use soap elements 101 are arranged in a multilayered concentric pattern to form a representation of a flower. When one wishes to wash they remove one of the soap elements, e.g., flower petals, and wash with the soap element 101. After washing the residual soap element, if any, is then discarded. FIG. 2 illustrates a top view of one example of a soap assembly described herein comprising a plurality of soap element 201. FIG. 3 provides a bottom view of a soap assembly having base 310 and soap elements 301. The illustration shows the overlapping multilayered concentric arrangement of the outer levels of the assembly. FIG. 4 shows a side view of one example of a soap assembly described herein having base 410 and soap elements 401.

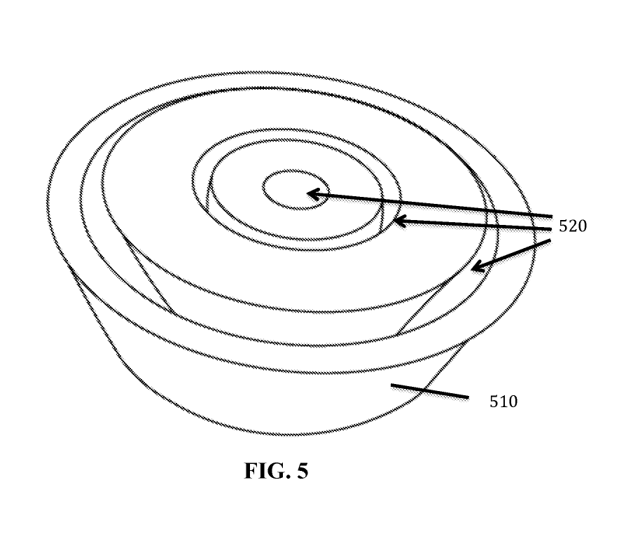

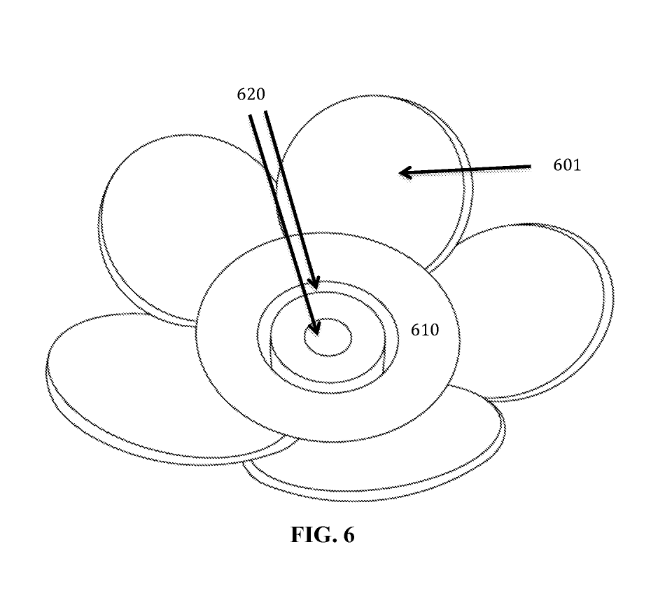

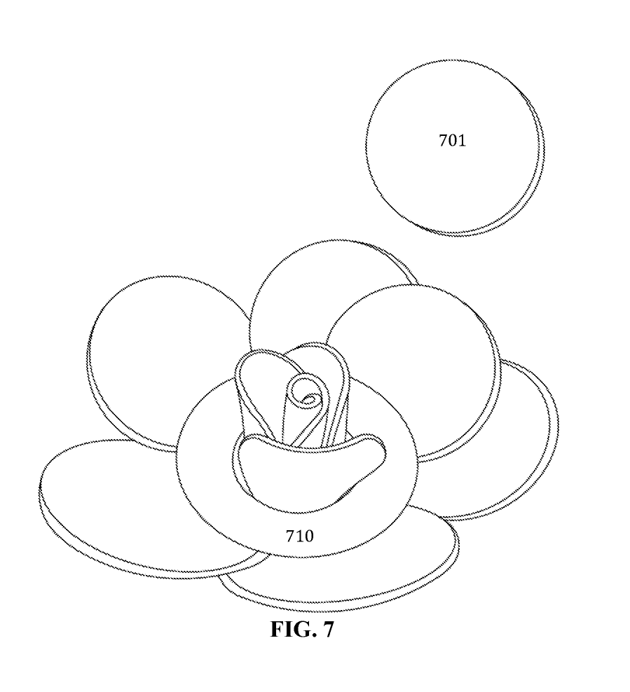

FIG. 5 shows one example of base 510 having grooves 520 for positioning the soap elements. In this particular example the base is circular, but can be any geometric shape, e.g., an oval, triangle, square, rectangle, pentagon, octagon, or other regular or irregular polygons. FIG. 6 shows the initial steps in constructing the assembly by positioning a first layer of soap elements 601 around a groove 620 in base 610. FIG. 7 shows the assembly the center portion, which can be assembled before or after the assembly of the first outer layer. Additional layers of soap elements 701 are assembled by positioning other layers of soap element 701 in base 710 layers between the outer layer and the center portion of the assembly.

In certain aspects single use soap elements can be prepared by processing soap formulations containing soap-forming components. In certain aspects the components or formulations can be processed by melting the soap formulation and pouring or molding the melted soap. After pouring or molding the soap is left for a period of time to solidify or set. Once solidified the soap elements can be shaped or bent as needed for constructing the assembly. In other aspect the soap composition can be pressed or cut into the shape of a single use soap element. The single use soap element can be approximately circular elliptical or any other geometric shape desired. In a particular aspect the soap elements are molded or pressed into a shape approximating a flower petal.

Any of the usual soap-forming mixtures currently used to produce a translucent or opaque soap elements can be used. The soap can be colored, texture, or contain inclusions. Color can be attained by using an appropriate pigment or combination of pigments. Pigments can include carbon black, and pearlescent pigments which contain black metal oxides as partial or total coatings on mica can be used. These include black iron oxide (Fe3 O4), ilmenite (FeTiO3), manganese dioxide, and other readily available and non-toxic black pigments. Many different organic colorants can be used to obtain the color effects in soap bars. Pigments can include phthalocyanine blue, phthalocyanine green, quinacridone red and carbazole violet. Also, absorption colorants similar in structure or chemistry can also be used, as well as others of equal or similar stability properties. Inclusion can be abrasives or perfumes or moisturizers. In certain aspects the soap comprises one or more of propylene glycol, sorbitol, water, sodium stearate, sodium laureth sulfate,sodium laurate, glycerin, titanium dioxide, pigment, and fragrance (e.g., fragrance oil).

1. A soap assembly comprising a base and a plurality of single use soap elements, wherein the base forms grooves to align the single use soap elements into a decorative assembly.

2. The soap assembly of claim 1, wherein the single use soap elements are in the shape of flower petals.

3. The soap assembly of claim 1, wherein the decorative assembly is in the shape of a flower.

4. The soap assembly of claim 3, wherein the flower is a rose.

5. The soap assembly of claim 1, wherein the single use soap elements are approximately 1 to 6 cm in diameter.

6. The soap assembly of claim 1, wherein the single use soap elements are about 0.5 to 10 millimeters thick.
